(19) United States
(12) Patent Application Publication
(10) Pub. No.: US 2007/0041925 A1
(43) Pub. Date: Feb. 22, 2007

(54) Skin and hair care preparation containing a combination of protein hydrolyzates

(75) Inventors: Roger Picano, Seerzach (CH); Sven Gohla, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/572,751

(22) PCT Filed: Sep. 11, 2004
(86) PCT No.: PCT/EP04/10185

§ 371 Date: Oct. 4, 2006

(30) Foreign Application Priority Data
Sep. 11, 2004 (DE) 103 44 166.2
Nov. 20, 2003 (DE) 103 54 316.3

(51) Int. Cl. A61K 8/97; A61K 8/64

(52) U.S. Cl. 424/70.14; 424/74

(57) ABSTRACT

The invention relates to cosmetic preparations, especially hair care preparations or hair care products, containing protein hydrolysates of silk, pashmina, cashmere wool, merino wool and/or mohair, mussel thread extracts, so-called bysuss threads, and sericin and/or sericin hydrolysates. The preparations are mild for skin and hair and lead to an improvement of hair structure and physical-optical hair properties.

The invention relates to cosmetic preparations, in particular hair care preparations or hair care compositions, comprising protein hydrolyzates from silk, pashmina, cashmere wool, merino wool and/or mohair, extract from the mussel threads of mussels, the so-called byssus threads, and sericin and/or sericin hydrolyzates. The preparations are mild to skin and hair and lead to an improvement in the hair structure and also in the physical-optical hair properties.

The entire human body, with the exception of the lips, the palms of the hands and the soles of the feet, is covered in hair, albeit for a large part with barely visible down. Because of the many nerve endings at the hair root, hair reacts sensitively to external influences such as wind or touch and is therefore a component of the sense of touch that should not be underestimated. The most important function of human head hair must, however, nowadays be in helping to create the appearance of the person in a characteristic manner. Similarly to the skin, it fulfils a social function since, via its outward appearance, it contributes considerably to interpersonal relations and to the self-esteem of the individual.

The hair consists of the hair shaft which protrudes freely from the skin—the keratinized (dead) section which represents the actual visible hair—and the hair root which sticks in the skin—the living section, in which the visible hair is continually renewed. The hair shaft is in turn made up of three layers, a central section—the so-called hair marrow (medulla), although in humans this has retrogressed and is often missing altogether—also the marrow (cortex) and the outer horny layer which is up to ten layers thick (cuticle), which surrounds the entire hair.

Provided there are no pathological changes, it is virtually impossible to improve upon human hair in its freshly grown state. The section of the hair in the vicinity of the scalp accordingly has a virtually closed horny layer. In particular, the horny layer, being the external sheath of the hair, but also the inner region below the cuticle are subjected to particular stress by environmental influences.

Significant influences for the loss in quality of a hair during its aging are the influence of sunlight, mechanical stresses by intensive combing or brushing, but also hair treatments, such as hair colorations and in particular, bleaching, and also hair shaping, for example permanent waving methods. Accordingly, oxidative stresses in particular often lead to hair damage.

Both UV-A and also UV-B radiation have a harmful effect on the hair, which is evident, for example, from the fact that certain amino acids, such as cystine and methionine, are degraded, or sulfur-sulfur bonds of the keratin are cleaved, which, in the worst case scenario, can result in destruction of the hair. Furthermore, hair and scalp are parts of the body which, on account of their position, are exposed to a considerable amount of UV radiation when out and about outdoors.

One aim of hair care and thus also of the present invention is to retain the natural state of freshly grown hair over as long a period as possible and, in the case of loss, to restore it. A silky shine, low porosity and a pleasant, smooth feel are features of natural healthy hair.

Since the end of the previous century, hair care products have been developed specifically. This led to a large number of preparations both for general hair care and also for overcoming the anomalies of hair and of the scalp. In general, use is nowadays made of hair care cosmetics which, after they have worked, are either intended to be washed out of the hair again, or which should remain on the hair. The latter can be formulated such that they not only serve to care for the individual hair, but also improve the appearance of the hairstyle overall. Hair groomed in such a way is characterized by a pleasant feel, natural shine, increased fullness, suppleness and thus good styleability and hold and thus good hairstyle sit.

Products which serve exclusively to care for the hair are generally referred to as hair conditioning agents or conditioners. After a greater or lesser residence time on the hair, these can be rinsed out (rinse-off products, e.g. rinses, hair treatments) or they remain following application to the hair (leave-on products). The products can have various consistencies, meaning that they can be applied in very different ways. They may be emulsions or gels or low-viscosity solutions which are applied, for example, by means of spray applications, or foams, which are generated, for example, by suitable pressurized-gas packaging or a special foam pump during application. Creamy, opaque and clearly transparent products can be found on the market.

Depending on the intended use, quite diverse active ingredients or combinations of active ingredients are found in such conditioners. Some, which serve more to protect the hair, such as antioxidants or UV filters, others which make the hair supple, such as, for example, cationic surfactants. Achieving ever greater importance are polymeric active ingredients which have very diverse properties depending on their nature, molecular weight and charge. However, an improvement in the nature of the surface of the hair is clearly at the forefront.

Despite a great diversity of products which are available to the consumer, some disadvantages have still not been completely overcome. For example, polymers which give the hair a certain hold and thus volume often exhibit a poor feel sensation and poor combability; polymers which make the hair supple often lead to it being weighed down, which is associated with lack of volume. The use of starch derivatives has in the past also been unable to overcome the described disadvantages of the prior art.

Gel-like preparations of the prior art also have a number of other disadvantages: in most cases carbomers, i.e. polyacrylates or cellulose derivatives are used for thickening the preparations. However, such preparations usually have a cloudy appearance. Virtually no salt-like care substances and no ionic film formers can be incorporated into the preparations of the prior art either since this leads to incompatibilities (substance deposits, precipitations, viscosity losses, etc.). The sensory and tactile properties of conventional preparations thickened with carbomers or cellulose derivatives also do not correspond to consumers' requirements since they are perceived as greasy and slippery.

It is also an object of the present invention to provide formulation variants which have improved sensory and tactile properties compared with preparations of the prior art. In particular, it was also to provide preparations whose biopolymer constituents can be released to a considerable degree from the formulations for hair and scalp care.

Hair washing also plays a central role in human body care. The cleansing of the hair and of the scalp of grease produced by the body, skin peeling, dirt and odors is a basic human requirement.

To satisfy this requirement, until into the 20th century, man only had soap at his disposal which, on account of its alkaline pH, was not well tolerated by the scalp and the eye mucosa and often left behind deposits of lime soaps in the hair. The 1930s saw the first alkyl sulfate-containing shampoo. Since the middle of the 1960s, alkyl ether sulfates and other surfactants have captured the shampoo market. Using these, it is possible to avoid the disadvantages of soap-containing preparations. Nowadays modern shampoos not only have to cleanse the hair and be well tolerated, but they should also care for the hair and increase its styleability and optical attractiveness.

Hair shampoos comprise a large number of different components in order to meet the individual requirements placed on the product.

The cleaning power of the shampoo is brought about by the presence of anionic, amphoteric and nonionic surfactants as surface-active compounds in the preparations. Moreover, surfactants ensure the foaming ability of the hair-cleansing compositions. Moreover, when choosing the surfactants, their insensitivity to water hardness, their biodegradability, their compatibility with other components of the preparation and also their price are important. A much-used shampoo surfactant is, for example, alkyl ether sulfate.

Moreover, shampoos comprise a series of consistency regulators which give the preparation the desired viscosity. These thickeners bring about an enlargement of the surfactant micelles and/or swelling of the water phase of the preparation. Thickeners can be chosen from classes of substances which are chemically very different. For example, electrolytes (e.g. sodium chloride), alkanolamides (e.g. fatty acid monoethanolamide), fatty alcohols with low degrees of ethoxylation (e.g. diethylene glycol monolauryl ether), ethers with high degrees of ethoxylation, esters and diesters, and polymeric thickeners, inter alia, are used. The polymeric thickeners include, for example, cellulose ethers. Moreover, polyacrylates and hydrocolloids are also used as thickeners. Polymeric thickeners have the major advantage that the viscosity produced by them is largely temperature-independent.

Besides perfumes and dyes and a series of compounds which increase the shelf-life of the preparations, different types of active ingredients have been added to hair shampoos in more recent times. Besides UV absorbers, vitamins or plant extracts, these also include so-called hair conditioners, which care for the hair and improve its combability and its feel, and increase its shine. In contrast to most other constituents of shampoos, conditioners attach to the hair and remain there after rinsing. On account of their molecular structure, they deposit themselves at the damaged sites of the hair cuticle and smooth the hair. As a result, the hair becomes less rough and brittle, the hairstyle is given considerably more shine and can be combed more easily. The hair is also less sensitive to electrostatic charging. The most important hair-conditioning substances, generally cationic polymers, are the polymeric quaternary ammonium compounds. Cationic cellulose derivatives and polysaccharides can also be used. Furthermore, silicone compounds are also used for conditioning.

However, hair washing with aggressive surfactants can also stress the hair, at least reduce its appearance or the appearance of the hairstyle overall. For example, certain water-soluble hair constituents (e.g. urea, uric acid, xanthine, keratin, glycogen, citric acid, lactic acid) can be leached out as a result of hair washing.

The prior art lacks shampoo formulations which care for damaged hair in a satisfactory way. It is therefore an object to also overcome these disadvantages of the prior art.

The object of the present invention is therefore also to provide an alternative cosmetic cleansing and care preparation which is characterized in particular by good skin and hair compatibility, good foaming behavior and pleasant hair care and styling.

This bundle of objects is achieved by a cosmetic and/or dermatological preparation corresponding to the main claims. Preferred embodiments of the preparation are disclosed in the dependent claims. Moreover, the invention also covers the use of the preparations on the skin and/or the hair.

It has surprisingly been found, and herein lies the attainment of these objects, that a cosmetic preparation comprising
a.) protein hydrolyzates from silk, pashmina, cashmere wool, merino wool and/or mohair,
b.) extract from the mussel threads of mussels, the so-called byssus threads, and
c.) sericin and/or sericin hydrolyzates and optionally
d.) additionally biogenic and/or functional amino acids, such as carnithine, creatine, taurine and/or glutathione
   perceptibly and visibly improves the skin and hair properties.

In this connection, the interplay and the resulting synergistic effect of the special protein hydrolyzates from the biogenic fibers, such as, for example, pashmina or merino wool, silk and mussel byssus with osmolytic amino acids, e.g. taurine, for hair care, skin care and, in particular, scalp care is to be emphasized.

In particular, the combinations of a.) cashmere wool preparations, b.) byssus silk preparations and c.) silk bast sericin, and optionally the biogenic amino acids d.) referred to above obtain a clearly detectable improvement in the hair structure and sensory properties.

The combination according to the invention of the constituents a.-c. and optionally d. shows a synergism which is responsible for the surprisingly extremely positive properties of the cosmetic preparation. The individual constituents are bound in a cosmetic preparation to give an active ingredient complex and only develop their advantageous positive properties on the hair and the scalp in the composition according to the invention.

In internal studies, it was shown that the preparations according to the invention bring about a tactile advantage, in particular with regard to hair fullness, suppleness and shine. These advantages are not observed through cosmetic preparations which each comprise one or two of the individual components according to the invention.

The combinations according to the invention stimulate the metabolism in the scalp and promote the moisture enrichment on the scalp and in the hair and are characterized in particular by good skin and hair compatibility. In addition, the amino acid constituents deliver the factors back to skin and hair which, like “magnets”, lead to the ideal moisture in the skin and its appendages. This has a positive effect particularly on the styleability of the hair, which threatens to lose its styleability as a result of electrostatic charging as a result of too little moisture or moisture which is too rapidly volatile.

As a result of the topical application of the preparation on the skin and/or the hair, the following positive properties of the treated hair and/or skin, in particular, are found:
improvement in the styleability of the hair,
avoidance of the negative effect of static charging of the hair,
stabilizing effect on the hair structure,
caring for hair and scalp, particularly over a prolonged period following application,
structure-smoothing effect,
imparts natural shimmer and attractive volume to the hair,
the hair is easier to comb and easier to style,
protects the hair and the scalp during blow-drying,
protects against electrostatic charging by balancing and equalizing the moisture level in the hair.

Preferably, the preparations according to the invention are prepared and sold in silicone-free form.

As shampoo, surprisingly positive properties were also found, thus the hair treated with the preparations according to the invention looks extremely well cared for and can be styled easily. This is evident, for example from the combability, the appearance and the fullness of the hair.

The constituent a.) according to the invention is chosen from protein preparations from silk, pashmina, cashmere wool, merino wool and/or mohair.

Pashm is the Persian word for wool and pashmina and describes the fine, fleecy pashmina wool which originates and is obtained from the heights of the Tibetan Himalaya. As cosmetic product, Pashmisilk®, for example is known, comprising a hydrolyzate from pashmina, merino wool and natural silk.

Protein hydrolyzates of cashmere wool have already been described. They are manufactured and sold, for example by Laboratoires Serobiologiques, France and sold as “Cashmilan”.

Protein hydrolyzates of merino wool or mohair wool are likewise intended as constituents essential according to the invention. Mohair wool is obtained from the angora goat.

The protein hydrolyzate from pashmina, cashmere wool, merino wool and/or mohair essentially comprise amino acid, peptide and keratin hydrolyzates.

Preference is given to using hydrolyzed silk protein, for example as Silkpro® from Ikeda Corp. (CAS No.: 96690-41-4).

Known to a lesser degree is the fact that some types of mussel produce raw material for cosmetics. For example, mussel silk or byssus silk, also called golden sea wool or sea silk, was in the past raw material for fashion, the legendary “Golden Fleece”. However, the word “byssos” applies to different fine fabrics, meaning that only with additional designations or the circumstances can mussel silk be inferred.

For example, Mythuline®, is known, an extract of the mussel threads of mussels produced by Laboratoires Seporga. The extract contains microfibers with alternating groups of collagen and elastin which are held together by fibroin, as the diagram below shows.

[image]

The most significant subspecies of mussels are the North Sea mussels, Mediterranean mussels, sea dates and New Zealand mussels. All of these types are found predominantly in shallow water where, with the help of their byssus, they cling to rocks or to one another. The name mussels arose from this byssus, also the so-called moss, which they use as tentacles, and which, as a result of a shift in sound, gives not moss, but mussels.

In the course of our life, many small movements cause great mechanical stress to our skin and hair. The repetition of these stresses together with the deorganization of the connective fibers (glycolization, elastin degradation, collagen polymerization) leads to a loss in the elasticity and firmness of the skin, accelerates the aging process and thus wrinkling. Mussels have to overcome very similar physical stress situations to our skin and our hair. Their threads have to ensure firm hold to the substrate in order to be able to withstand the changing conditions of their environment (waves, becoming dry, fighting for space with other mussels, algae). Since nature can solve problems in exemplary fashion, the mussel thread of Mytilus edulis, the mussel, consists of three strong protein micro-fibers (elastin, exocollagen, fibroin) whose specific structure and biomechanical properties of the fibers simultaneously impart resistance and elasticity.

Here, elastin and exocollagen peptide alternate within the thread. The filament is completely covered by fibroin (silk from the sea). As a result of the refraction of light at their surface, the silk fibers also appear shiny. Fibroin is the essential constituent of silk, a thread-like, flexible and lustrous substance. Similarly to a prism, it gives the silk the characteristic silky effect. The main effect of the silk threads is to ensure the resistance and elasticity of the mussel threads.

Elastin can be stretched up to 160% of its length without tearing. Elastin is co-responsible for the elasticity of the mussel threads.

An extract of these threads, referred to as mussel threads of mussels, the byssus threads, leads, in combination with the constituents a.) and c.) according to the invention to a moisturizing and strengthening effect of the hair. In addition, the synthesis of collagen I and III is increased.

The additional effect of the silk shimmer improves the shine of the hair.

Sericin has proven to be a third essential constituent of the preparations according to the invention. Sericin is a protein substance from the cocoon of the silk spider which surrounds the raw silk thread. For example, Setakol®, a hydrolyzate of the silk protein sericin, which is manufactured and sold by Pentapharm, Basle, is known. This is an aqueous solution of the silk protein sericin. The advantageous properties of sericin which are used according to the invention in combination with the properties of the constituents a. and b. is that sericin binds with creatine in the skin and the hair and thus enables a protective effect. Sericin thus leads to a protective function for the hair or skin treated with the preparation according to the invention.

Preferably, the preparations according to the invention comprise Pashmisilk®, Silkpro® and/or “Cashmilan” as constituent a. according to the invention, Mythulinee as constituent b. and Setakol® as constituent c. These three protein preparations each constitute hydrolyzates of the individual constituents a., b. and c. preferred according to the invention. The combination leads to a significantly improved softness and suppleness of the hair. In addition, the shine of the hair is significantly improved, and by anchoring moisturizing factors in the hair, the styleability is significantly improved.

The preparation preferably comprises an amount of from 0.001 to 50% by weight, based on the total mass of the preparation, in particular, 0.01 to 15%, preferably 0.05 to 5%, of functional or biogenic amino acids, preferably with the amino acids taurine and/or creatine.

The weight fractions of components a.-d. among one another can preferably assume the following ranges.

a. 0.025-99% by weight, preferably 0.5-50% by weight

b. 0.025-99% by weight, preferably 0.5-50% by weight

c. 0.025-99% by weight, preferably 0.5-50% by weight

d. 0-50% by weight, preferably 0.01-15% by weight.

On account of the constituents obtainable from the various protein preparations, it has proven particularly preferred for the cosmetic preparations to comprise a combination of HYDROLYZED COLLAGEN (constituent b.), CAS No. 92113-31-0), HYDROLYZED ELASTIN (constituent b.), CAS No. 100085-10-7), HYDROLYZED KERATIN (constituent d.), CAS No. 69430-36-0), HYDROLYZED SERICIN (constituent c.), CAS No. 96690-41-4) and HYDROLYZED SILK (constituent a.), CAS No. 73049-73-7).

Particular preference is given to a cosmetic preparation comprising 0.005-0.03% by weight of HYDROLYZED COLLAGEN, 0.005-0.03% by weight of HYDROLYZED ELASTIN, 0.05-0.5% by weight of HYDROLYZED KERATIN, 0.01-0.5% by weight of HYDROLYZED SERICIN and 0.01-0.5% by weight of HYDROLYZED SILK.

Preferably, the preparation according to the invention additionally comprises taurine. Through the addition of taurine, its osmolyte effect is utilized, which brings about an increase in the amount of moisture on the scalp and the hair. This additional effect leads to a further improvement in the styleabilty of the hair.

Through the addition of taurine in the preparation according to the invention, an increase in moisture of the scalp and/or of the hair is established.

In particular, through the addition of taurine it is possible to avoid the annoying effect of static charging of the hair.

In this connection, this invention provides that surprisingly, in particular, products or preparations of products of biogenic amino acids, for example, taurine, together with preparations of sea silk, pashmina and silk bast sericin are to be regarded as being particularly advantageous.

Taurine is to a limited extent an essential amino acid which is found in the tissues of most types of animal. It does not serve as a building block of proteins but is present in many tissues in free form. Taurine is involved in a series of physiological processes, e.g. the conjugation of bile acids, osmoregulation, detoxification of xenobiotics, stabilization of cell membranes, control of the cellular calcium stream and modulation of neuronal excitability. Taurine (2-aminoethanesulfonic acid) differs from other amino acids in that the carboxyl group is replaced by a sulfonic acid group and in that it is not incorporated into proteins. Taurine is thus not an amino acid in the narrower sense.

The production or provision of constituents a.-d. is known to the person skilled in the art. The cosmetic and dermatological preparations according to the invention can comprise cosmetic auxiliaries as are customarily used in such preparations.

For example, the cashmere hydrolyzate can be obtained by enzymatic hydrolysis of cashmere wool, subsequent filtration and preservation.

The extract from mussel threads, the so-called byssus threads, is preferably obtained from the mussel Mytilus edulis. The mussel threads are hydrolyzed for several hours at at least 70° C. in an acidic aqueous solution. The pH is adjusted to 5-6 and the liquid product is decolored by filtering with activated carbon and then centrifuged. The product is then sterilized by means of sterile filtration and stored for 12 h at 65° C.

An additional content of antioxidants is generally preferred in cosmetic and/or dermatological preparations.

The compositions according to the invention can, for example, be in the form of preparations sprayable from aerosol containers, squeezable bottles or by a pump, spray or foaming device, but also in the form of a composition which can be applied from standard bottles and containers.

For the purposes of the present invention, suitable propellants for cosmetic or dermatological preparations which are sprayable from aerosol containers are the customary known, readily volatile, liquefied propellants, for example dimethyl ether, hydrocarbons (propane, butane, isobutane), which can be used on their own or in a mixture with one another. Compressed air, nitrogen, nitrogen dioxide or carbon dioxide or mixtures of these substances are also to be used advantageously.

The person skilled in the art is of course aware that there are propellant gases which are nontoxic per se, which would in principle be suitable for realizing the present invention in the form of aerosol preparations, but which nevertheless have to be dispensed with due to an unacceptable effect on the environment, or other accompanying circumstances, in particular fluorinated hydrocarbons and chlorofluorocarbons (CFCs).

Hair care compositions refers to a large number of products, the most important representatives of which are pretreatment compositions, hair tonics and hair treatment compositions.

Basic substances for hair care compositions are, for example, fatty alcohols, waxes, paraffins, Vaseline, paraffin oil and solvents.

Fatty alcohols are, for example, straight- or branched-chain aliphatic monohydric alcohols having 6-22 carbon atoms in the molecule. In cosmetics, preferably straight-chain fatty alcohols with a chain length of 12-18 carbon atoms are used. These fatty alcohols are soft, colorless masses, virtually nontoxic and well tolerated by the skin. Fatty alcohols are preferably used for producing hair treatments and styling creams, with cetyl alcohol and stearyl alcohol being attributed particular importance.

Waxes are fatty acid esters which occur in animal and vegetable products, but can also be produced synthetically. Probably the best known naturally occurring wax is beeswax, which comprises ceresin and myricin as main constituents. However, wax is a generic term for a series of naturally or synthetically obtained substances which are generally semi-solid, white, odorless masses which are insoluble in water.

In the cosmetics sense, paraffins are white, odorless masses of straight-chain high molecular weight hydrocarbons. Due to their properties being comparable with those of waxes, they are often also referred to as petroleum waxes.

Vaseline is a mixture of branched-chain paraffins with a low content of cyclic paraffins. It is a soft, transparent mass which is insoluble in water and has a low intrinsic odor which is produced during petroleum processing.

Paraffin oil is a mixture of saturated liquid hydrocarbons. It is insoluble in water, but miscible with fatty alcohols and waxes. It is used as additive in hair color compositions for regulating consistency.

Solvents play a considerable role in cosmetics. Of the large number of solvents which are available, ethanol is of greatest importance. It is used for producing hair tonics in which, on account of its disinfecting properties, at the same time fulfils the function of an active ingredient.

Auxiliaries can be used in order to improve certain properties of hair care compositions, e.g. consistency, thermal stability and photostability, appearance and odor, and to facilitate their production. For example, the following are added as required:
emulsifiers, in order to reduce the interfacial tension between two immiscible phases so much that their fine mixing is possible,
thickeners in order to increase the stability of the emulsions and to adjust their viscosity,
UV absorbers in order to improve the photostability of the dyes present in hair care compositions and other photosensitive components. In addition, they serve to protect the hair against the effects of light.
Preservatives, in order to prevent microbial decomposition.
Antioxidants, in order to prevent changes in odor which can be caused by oxidation processes.
Dyes, in order to impart a pleasing appearance to hair care compositions.
Perfume oils in order to impart a pleasant odor to hair treatment compositions and to conceal secondary odors of the raw materials.

Quaternary ammonium compounds are an important group of the special active ingredients which are used for producing hair care compositions. Hair care compositions, in particular hair treatments, are given essential properties such as improving combability and feel and preventing static charging of the hair, primarily through the use of quaternary ammonium compounds. This property can additionally be improved through the content of taurine.

The properties of quaternary ammonium compounds are determined by the cationic group on the one hand and by the type of lipophilic radicals of this group on the other hand. Of suitability are, for example, the compounds in which one to two radicals are longer-chain alkyl groups, such as lauryl, cetyl or stearyl groups, and the remaining radicals are methyl groups. Products of this type are preferably used as chlorides, bromides and methosulfates.

Also suitable are polymeric quaternary ammonium compounds, macromolecules whose essential feature is the presence of a plurality of quaternary ammonium groups in the molecule. As a result, their ability to adhere to the hair is considerably increased.

Cationic surfactants to be used particularly advantageously are

1. alkylamines

2. alkylimidazoles

3. ethyoxylated amines and

4. quaternary surfactants

5. ester quats.

Quaternary surfactants contain at least one N atom which is covalently bonded to 4 alkyl or aryl groups. Irrespective of the pH, this leads to a positive charge. The cationic surfactants used according to the invention can also preferably be chosen from the group of quaternary ammonium compounds, in particular benzyltrialkylammonium chloride, also alkyltrialkylammonium salts, for example cetyltrimethylammonium chloride or bromide, alkyldimethylhydroxyethylammonium chlorides or bromides, dialkyldimethylammonium chlorides or bromides, alkylamide ethyltrimethylammonium ether sulfates, alkylpyridinium salts, for example lauryl- or cetylpyrimidinium chloride, imidazoline derivatives and compounds with cationic character, such as amine oxides, for example alkyldimethylamine oxides or alkylaminoethyldiemthylamine oxides. In particular, cetyltrimethylammonium salts are to be used advantageously.

Monomeric or polymeric quaternary ammonium compounds are used widely in hair rinses and hair treatments, e.g. in concentrations of 0.5-5% by weight. These include cetrimonium chloride, as is supplied under the name Dehyquart A by Henkel, or distearoylethylhydroxyethylmonium methosulfate, as is supplied under the name Dehyquart F 75 by Henkel.

If the cosmetic or dermatological preparations are present in the form of a lotion which is rinsed out and used, for example, before or after bleaching, before or after shampooing, between two shampooing steps, before or after permanent wave treatment, then these are here, for example, emulsions which optionally comprise surface-active substances whose concentration can be between 0.1 and 10% by weight, preferably between 0.2 and 5% by weight.

A cosmetic preparation in the form of a lotion which is not rinsed out, in particular a lotion for arranging the hair, a lotion which is used while blow-drying the hair, a styling and treatment lotion, is generally an emulsion and comprises the combinations according to the invention. However, it is in some cases advantageous if the lotion according to the invention is in the form of a microemulsion for an aqueous or aqueous-alcoholic solution.

According to the invention, cosmetic preparations for the treatment and care of the hair can be in the form of gels which comprise organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates, such as, for example, bentonites, or a mixture of polyethylene glycol and polyethylene glycol stearate or distearate. The thickener is present in the gel e.g. in an amount between 0.1 and 30% by weight, preferably between 0.5 and 15% by weight.

The percentages given above refer to the total weight of the preparations.

The preparations according to the invention can comprise cosmetic auxiliaries, as are customarily used in such preparations, e.g. preservatives, perfumes, substances for preventing foaming, foam stabilizers, dyes, pigments which have a coloring effect, thickeners, surface-active substances, emulsifiers, softening, moisturizing and/or humectant substances, re-fatting agents, fats, oils, waxes, alcohols, polyols and the toxicologically compatible ethers and esters thereof, branched and/or unbranched hydrocarbons, further antioxidants, stabilizers, pH regulators, consistency regulators, bactericides, deodorants, antimicrobial substances, antistats, UV absorbers, complexing and sequestering agents, pearlescent agents, polymers, electrolytes, organic solvents, silicone derivatives, plant extracts, vitamins and/or other active ingredients or other customary constituents of a cosmetic or dermatological formulation. Solubility promoters, e.g. for incorporating hydrophobic components, such as, for example perfume preparations, may also be present.

The total amount of the auxiliaries is, for example, 0.001 to 15% by weight, preferably 0.01 to 10% by weight, in each case based on the total weight of the preparation.

The amount of thickeners is, for example, 0.05 to 5.0% by weight, preferably 0.1 to 3.0% by weight, in particular 0.15 to 2.0% by weight, in each case based on the total weight of the preparation.

The water content of the preparations is, for example, 60 to 95% by weight, preferably 75 to 95% by weight, in particular 80 to 90% by weight, in each case based on the total weight of the preparation.

To protect the formulation and/or the hair to be treated, all antioxidants which are customary or suitable for cosmetic and/or dermatological applications can be used.

The total amount of the antioxidants is, for example, 0.001 to 2% by weight, preferably 0.01 to 1% by weight, in each case based on the total weight of the preparation.

Further antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximine, homocysteine sulfoximine, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and derivates thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

Furthermore, preparations according to the invention can advantageously comprises substances which absorb UV radiation in the UV-B region, where the total amount of the filter substance is, for example, 0.001% by weight to 30% by weight, preferably 0.05% to 10% by weight, in particular 0.1 to 1.0% by weight, based on the total weight of the preparations in order to provide cosmetic preparations which protect the hair and/or the skin from the entire range of ultraviolet radiation. They can also be used as sunscreens for the hair or the skin, in particular the scalp.

If the emulsions according to the invention comprise UV-B filter substances, these may advantageously be water-soluble. Advantageous water-soluble UV-B filters are, for example:
salts of 2-phenylbenzimidazole-5-sulfonic acid and its sodium, potassium or triethanolammonium salt, and the 2-phenylbenzimidazole-5-sulfonic acid itself;
sulfonic acid derivatives of benzophenones, preferably 2-hydroxy-4-methoxybenzophenone-5-sulfonic acid and its salts;
sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and its salts.

It may also be advantageous to add UV-A filters which have hitherto usually been present in cosmetic preparations to preparations according to the invention. The amounts used for the UV-B combination can be used.

The preparations according to the invention can be prepared in the usual manner by mixing the individual constituents. The active ingredients of the combinations according to the invention or else the premixed constituents of the combinations according to the invention can be added in the mixing operation.

The pH of the preparations can be adjusted in a known manner by adding acids or bases, preferably by adding buffer substances, e.g. based on citric acid/citrate or phosphoric acid phosphate buffer mixtures. The pH is preferably below 10, e.g. in the range 2-7, in particular in the range 3-5.

The examples below are intended to illustrate the present invention without limiting it. Unless stated otherwise, all amounts, proportions and percentages are based on the weight and the total amount or on the total weight of the preparations.

Protein hydrolyzates from silk, pashmina, cashmere wool, merino wool and/or mohair, extract from the mussel threads of mussels, the so-called byssus threads, and sericin and/or sericin hydrolyzates and optionally

### EXAMPLES

### Example 1

### Cleansing Gel

| | % by wt. |
|---|---|
| Protein hydrolyzate (silk, pashmina, | 5.00 |
| byssus threads, sericin) | |
| Sodium laureth sulfate | 13.00 |
| Sodium cocoamphoacetate | 2.00 |
| Citric acid | 0.50 |
| Dicaprylyl ether | 8.00 |
| Glyceryl linoleate | 2.50 |
| Glycerin | 5.00 |
| PEG-150 Distearate | 0.82 |
| Sodium chloride | 1.62 |
| Antioxidants | q.s. |
| Preservative | q.s. |
| Water | ad 100.000 |

### Example 2

### Bath Preparation

| | % by wt. |
|---|---|
| Protein hydrolyzate (cashmere, byssus | 7.50 |
| threads, sericin) | |
| Sodium laureth sulfate | 13.00 |
| Cocoamidopropylbetaine | 2.00 |
| Citric acid | 0.50 |
| Dicaprylyl ether | 8.00 |
| Glyceryl linoleate | 2.50 |
| Glycerin | 5.00 |
| PEG-150 Distearate | 0.82 |
| Sodium chloride | 1.62 |
| Antioxidants | q.s. |
| Preservative | q.s. |
| Water | ad 100.000 |

### Example 3

### Shower Gel

| | % by wt. |
|---|---|
| Protein hydrolyzate (silk, pashmina, | 15.00 |
| byssus threads, sericin) | |
| Sodium laureth sulfate | 13.00 |
| Sodium cocoamphopropionate | 2.00 |
| Citric acid | 0.23 |
| Dicaprylyl ether | 8.00 |
| Glyceryl linoleate | 3.00 |
| Glycerin | 5.00 |
| PEG-150 Distearate | 0.85 |
| Sodium chloride | 1.63 |
| Antioxidants | q.s. |
| Preservative | q.s. |
| Water | ad 100.000 |

### Example 4

### Cleansing Preparation

| | % by wt. |
|---|---|
| Protein hydrolyzate (silk, merino wool, | 2.75 |
| pashmina, byssus threads, sericin) | |
| Sodium laureth sulfate | 9.00 |
| Sodium cocoamphopropionate | 6.00 |
| Citric acid | 0.40 |
| Dicaprylyl ether | 8.00 |
| Glyceryl linoleate | 2.50 |
| Glycerin | 5.00 |
| PEG-150 Distearate | 0.83 |
| Sodium chloride | 0.81 |
| Antioxidants | q.s. |
| Preservative | q.s. |
| Water | ad 100.000 |

### Example 5

### Cleansing Gel

| | % by wt. |
|---|---|
| Protein hydrolyzate (silk, merino wool, | 12.75 |
| pashmina, byssus threads, sericin) | |
| Sodium laureth sulfate | 13.00 |
| Disodium cocoylglutamate | 2.00 |
| Citric acid | 0.65 |
| Cococaprylate/caprate | 8.00 |
| Glyceryl linoleate | 2.50 |
| Glycerin | 5.00 |
| PEG-150 Distearate | 1.00 |
| Sodium chloride | 1.63 |
| Antioxidants | q.s. |
| Preservative | q.s. |

### Example 6

### Shampoo

| | % by wt. |
|---|---|
| Protein hydrolyzate (pashmina, byssus | 8.00 |
| threads, sericin) | |
| Sodium laureth sulfate | 9.00 |
| Sodium cocoamphoacetate | 6.00 |
| Citric acid | 1.20 |
| Paraffinum liquidum | 2.00 |
| Glyceryl linoleate | 1.00 |
| Glycerin | 5.00 |
| PEG-150 Distearate | 0.30 |
| Antioxidants | q.s. |
| Preservative | q.s. |
| Water | ad 100.000 |

### Example 7

### Foaming Shower Gel

| | % by wt. |
|---|---|
| Protein hydrolyzate (pashmina, byssus | 3.00 |
| threads, sericin) | |
| Sodium laureth sulfate | 9.00 |
| Sodium cocoamphoacetate | 6.00 |
| Citric acid | 1.20 |
| Paraffinum liquidum | 2.00 |
| Glyceryl isostearate | 1.00 |
| Glycerin | 5.00 |
| PEG-150 Distearate | 0.30 |

### Example 8

### Shampoo

| | % by wt. |
|---|---|
| Protein hydrolyzate (pashmina, byssus | 7.00 |
| threads, sericin, taurine, creatine) | |
| Sodium laureth sulfate | 9.00 |
| Sodium cocoamphoacetate | 6.00 |
| Citric acid | 1.20 |
| Paraffinum liquidum | 2.00 |
| Sorbitanisostearate | 1.00 |
| Glycerin | 5.00 |
| PEG-150 Distearate | 0.30 |

### Examples 9, 10, 11

### Conditioner Shampoo with Pearlescence

| | 9 | 10 | 11 |
|---|---|---|---|
| Protein hydrolyzate | | | |
| (Cashmere, silk, byssus | 5.5 | 4.5 | 9.0 |
| threads, sericin, taurine) | | | |
| Polymer JR 400¹ | 0.5 | 0.5 | 0.5 |
| Texapon K 14 S special² | 4.0 | 5.0 | 4.5 |
| Plantacare 2000³ | 9.0 | 8.0 | 8.5 |
| Pearlizing agent | 2.0 | 2.0 | 2.0 |
| Rewopol SBCS 50⁴ | 6.0 | 5.5 | 6.2 |
| Atlas G-4280⁵ | 3.2 | 3.5 | 3.0 |
| Preservative, perfume, | q.s | q.s | q.s |
| thickener, pH adjustment | | | |
| and solubilizer | | | |
| Water, completely de- | ad 100.0 | ad 100.0 | ad 100.0 |
| mineralized | | | |

### Examples 12-16

### PIT Emulsions

| | Examples | | | | |
|---|---|---|---|---|---|
| | 12 | 13 | 14 | 15 | 16 |
| Protein hydrolyzate | 15.0 | 8.5 | 2.0 | 17.0 | 12.0 |
| (Pashmina, silk, byssus threads, sericin) | | | | | |
| Glyceryl monostearate self-emulsifying | 0.50 | | 3.00 | 2.00 | 4.00 |
| Polyoxyethylene(12) cetylstearyl ether | | 5.00 | | 1.00 | 1.50 |
| Polyoxyethylene(20) cetylstearyl ether | | | | 2.00 | |
| Polyoxyethylene(30) cetylstearyl ether | 5.00 | | 1.00 | | |
| Stearyl alcohol | | | 3.00 | | 0.50 |
| Cetyl alcohol | 2.50 | 1.00 | | 1.50 | |
| 2-Ethylhexyl methoxycinnamate | | | | 5.00 | 8.00 |
| 2,4-Bis-(4-(2-ethylhexyloxy-)-2- | | 1.50 | | 2.00 | 2.50 |
| hydroxy)phenyl)-6-(4-methoxyphenyl)- | | | | | |
| (1,3,5)-triazine | | | | | |
| 1-(4-tert-Butylphenyl)-3-(4- | | | 2.00 | | |
| methoxyphenyl)-1,3-propanedione | | | | | |
| Diethylhexyl Butamindotriazone | 1.00 | 2.00 | | 2.00 | |
| Ethylhexyl Triazone | 4.00 | | 3.00 | 4.00 | |
| 4-Methylbenzylidene camphor | | 4.00 | | | 2.00 |
| Octocrylene | | 4.00 | | | 2.50 |
| Phenylene-1,4-bis(monosodium, | | | 0.50 | | 1.60 |
| benzimidazyl-5,7-disulfonic acid | | | | | |
| Phenylbenzimidazole sulfonic acid | 0.50 | | | 3.00 | |
| C12-15 Alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethylpolysiloxane | 2.00 | | | 0.50 | 0.50 |
| Shea butter | | 2.00 | | | 0.50 |
| PVP hexadecene copolymer | 0.50 | | | 0.50 | 1.00 |
| Glycerin | 3.00 | | 4.00 | 3.00 | 2.00 |
| Tocopherol acetate | 0.50 | | 0.25 | | 1.00 |
| Serinol | 2.00 | 5.00 | 1.00 | 2.00 | 3.00 |
| Lactic acid | 2.40 | 6.00 | 1.20 | | 3.60 |
| Taurine | | 0.3 | | 0.5 | |
| Sea salt | 5.00 | 2.50 | | | |
| alpha-Glucosylrutin | 0.10 | | 0.20 | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad | ad | ad | ad | ad |
| | 100 | 100 | 100 | 100 | 100 |

### Examples 17-21

### O/W Cream

| | Examples | | | | |
|---|---|---|---|---|---|
| | 17 | 18 | 19 | 20 | 21 |
| Protein hydrolyzate | 4.00 | 13.5 | 0.8 | 26.00 | 7.00 |
| (mohair, cashmere, silk, byssus threads, | | | | | |
| sericin) | | | | | |
| Glyceryl stearate citrate | | | 2.00 | | 2.00 |
| Glyceryl stearate, self-emulsifying | 4.00 | 3.00 | | | |
| PEG-40 stearate | 1.00 | | | | |
| Polyglycerol-3-methylglucose distearate | | | | 3.00 | |
| Sorbitan stearate | | | | | 2.00 |
| Stearic acid | | 1.00 | | | |
| Polyoxyethylene(20) cetylstearyl ether | | | | | |
| Stearyl alcohol | | | 5.00 | | |
| Cetyl alcohol | 3.00 | 2.00 | | 3.00 | |
| Cetylstearyl alcohol | | | | | 2.00 |
| C12-15 Alkyl benzoate | | | | | |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| 4-Methylbenzylidene camphor | | | 1.00 | | |
| 1-(4-tert-Butylphenyl)-3-(4- | | | 0.50 | | |
| methoxyphenyl)-1,3-propanedione | | | | | |
| Serinol | 5.00 | 4.00 | 5.00 | 1.00 | 2.00 |
| Lactic acid | | 4.80 | 6.00 | 1.20 | |
| Sea salt | 5.00 | | | | |
| Taurine | 0.3 | | 0.9 | 0.3 | |
| Betaine | | | | | 1.00 |
| Tocopherol | 0.1 | | | | 0.20 |
| Biotin | | | 0.05 | | |
| Ethylenediamine tetraacetic acid trisodium | 0.1 | | 0.10 | 0.1 | |
| Preservative | q.s. | q.s. | q.s. | q.s. | q.s. |
| Xanthan gum | | | | | |
| Polyacrylic acid | 3.00 | 0.1 | | 0.1 | 0.1 |
| Sodium hydroxide solution 45% | q.s. | q.s. | q.s. | q.s. | q.s. |
| Glycerol | | 1.00 | 1.00 | 5.00 | 3.00 |
| Butylene glycol | | 3.00 | | | |
| Perfume | q.s. | q.s. | q.s. | q.s. | q.s. |
| Water | ad | ad | ad | ad | ad |
| | 100 | 100 | 100 | 100 | 100 |

### Example 22

### Care Preparation

Protein Hydrolyzate

(pashmina, cashmere, silk, byssus threads, sericin) in the form of a mixture of Pashmisilk®, Silkpro®, Mythuline®, Setakol® and water.

### Example 23

### Shampoo

| | % w/w |
|---|---|
| BENZOIC ACID | 0.1375 |
| CALCIUM CHLORIDE | 0.0378 |
| CITRIC ACID | 0.5000 |
| DISODIUM PEG-5 LAURYLCITRATE | 2.2320 |
| SULFOSUCCINATE | |
| FRAGRANCE (PERFUME) | 0.6000 |
| HYDROLYZED COLLAGEN | 0.0166 |
| HYDROLYZED ELASTIN | 0.0167 |
| HYDROLYZED KERATIN | 0.1500 |
| HYDROLYZED SERICIN | 0.0700 |
| HYDROLYZED SILK | 0.0667 |
| METHYLPARABEN | 0.0038 |
| NIACINAMIDE | 0.1000 |
| ORYZANOL | 0.0500 |
| PANTHENOL | 0.3000 |
| PEG-200 HYDROGENATED | 0.4500 |
| GLYCERYL PALMATE | |
| PEG-3 DISTEARATE | 2.0000 |
| PEG-40 HYDROGENATED CASTOR | 0.3000 |
| OIL | |
| PEG-7 GLYCERYL COCOATE | 0.0500 |
| PHENOXYETHANOL | 0.0107 |
| POLYQUATERNIUM-10 | 0.5000 |
| SODIUM BENZOATE | 0.2950 |
| SODIUM CHLORIDE | 1.2000 |
| SODIUM COCOAMPHOACETATE | 2.1900 |
| SODIUM LAURETH SULFATE | 10.2180 |
| SODIUM SALICYLATE | 0.4000 |
| TAURINE | 0.1000 |
| TETRASODIUM IMINODISUCCINATE | 0.5000 |
| WATER (AQUA) | 77.5052 |

### Example 24

### Rich Mask

| | % w/w |
|---|---|
| CALCIUM CHLORIDE | 0.0378 |
| CETEARYL ALCOHOL | 6.0000 |
| CETRIMONIUM CHLORIDE | 1.0000 |
| FRAGRANCE (PERFUME) | 0.5000 |
| GLYCERIN | 3.8700 |
| HYDROLYZED COLLAGEN | 0.0249 |
| HYDROLYZED ELASTIN | 0.0251 |
| HYDROLYZED KERATIN | 0.2250 |
| HYDROLYZED SERICIN | 0.1050 |
| HYDROLYZED SILK | 0.1001 |
| LANOLIN | 2.0000 |
| LANOLIN ALCOHOL | 0.5000 |
| METHYLPARABEN | 0.2056 |
| NIACINAMIDE | 0.1000 |
| ORYZANOL | 0.0500 |
| PANTHENOL | 0.6000 |
| PHENOXYETHANOL | 0.0161 |
| PHYTANTRIOL | 0.1000 |
| PROPYLPARABEN | 0.1008 |
| SODIUM CITRATE | 0.0350 |
| TAURINE | 0.2000 |
| WATER (AQUA) | 84.2046 |

### Example 25

### Styling Spray

| | % w/w |
|---|---|
| CALCIUM CHLORIDE | 0.0151 |
| CETEARETH-20 | 0.1500 |
| CETRIMONIUM CHLORIDE | 0.0500 |
| CITRIC ACID | 0.0100 |
| FRAGRANCE (PERFUME) | 0.3000 |
| HYDROLYZED COLLAGEN | 0.0083 |
| HYDROLYZED ELASTIN | 0.0084 |
| HYDROLYZED KERATIN | 0.0750 |
| HYDROLYZED SERICIN | 0.0350 |
| HYDROLYZED SILK | 0.0334 |
| LAURTRIMONIUM CHLORIDE | 0.0075 |
| METHYLPARABEN | 0.0019 |
| NIACINAMIDE | 0.0200 |
| PANTHENOL | 0.3750 |
| PEG/PPG-15/15 DIMETHICONE | 1.0000 |
| PEG-40 HYDROGENATED CASTOR | 0.6000 |
| OIL | |
| PHENOXYETHANOL | 0.0054 |
| POLYQUATERNIUM-11 | 0.0600 |
| SODIUM BENZOATE | 0.2000 |
| SODIUM BISULFITE | 0.0150 |
| TAURINE | 0.0500 |
| VP/VA COPOLYMER | 3.7500 |
| WATER (AQUA) | 93.2300 |

### Example 26

### Conditioner

| | % w/w |
|---|---|
| ALPHA-ISOMETHYL IONONE | 0.0276 |
| BENZYL SALICYLATE | 0.0131 |
| BIS-DIGLYCERYL | 1.6000 |
| POLYACYLADIPATE-2 | |
| BUTYLPHENYL METHYLPROPIONAL | 0.0104 |
| CALCIUM CHLORIDE | 0.0378 |
| CETEARYL ALCOHOL | 2.0000 |
| CETRIMONIUM CHLORIDE | 1.0000 |
| CITRONELLOL | 0.0091 |
| COUMARIN | 0.0075 |
| EUGENOL | 0.0080 |
| FRAGRANCE (PERFUME) | 0.2970 |
| GERANIOL | 0.0113 |
| GLYCERIN | 3.8700 |
| HYDROLYZED COLLAGEN | 0.0083 |
| HYDROLYZED ELASTIN | 0.0084 |
| HYDROLYZED KERATIN | 0.0750 |
| HYDROLYZED SERICIN | 0.0350 |
| HYDROLYZED SILK | 0.0334 |
| HYDROXYISOHEXYL 3- | 0.0075 |
| CYCLOHEXENE CARBOXALDEHYDE | |
| HYDROXYPROPYL | 0.2500 |
| METHYLCELLULOSE | |
| LINALOOL | 0.0081 |
| METHYLPARABEN | 0.2527 |
| NIACINAMIDE | 0.1000 |
| ORYZANOL | 0.0500 |
| PANTHENOL | 0.3000 |
| PHENOXYETHANOL | 0.0054 |
| PROPYLENE GLYCOL | 0.5000 |
| PROPYLPARABEN | 0.1003 |
| SODIUM CITRATE | 0.0375 |
| TAURINE | 0.1000 |
| WATER (AQUA) | 89.2366 |

¹Cationic cellulose derivate from Amerchol,
²Myreth sulfate 70% from Cognis (AES)
³Decyl glucoside 50% from Cognis (APG)
⁴Citric acid alkyl polyglycol ester sulfosuccinate, disodium salt 38% from Goldschmidt (ACS)
⁵PEG-80 sorbitan laurate 73% from Uniqema (PSE)

1-11. (canceled)

12. A cosmetic preparation comprising
(a) a protein hydrolyzate from at least one of silk, pashmina, cashmere wool, merino wool and mohair,
(b) an extract from mussel threads of mussels,
(c) at least one of sericin and a hydrolyzate of sericin, and
(d) optionally, one or more amino acids which are at least one of biogenic and functional.

13. The preparation of claim 12, wherein the preparation comprises (d) and (d) comprises at least one of carnitine, creatine, taurine and glutathione.

14. The preparation of claim 13, wherein the preparation comprises taurine.

15. The preparation of claim 12, wherein the preparation comprises, based on a total weight of (a) to (d), from 0.025% to 99% by weight of (a), from 0.025% to 99% by weight of (b), from 0.025% to 99% by weight of (c) and from 0% to 50% by weight of (d).

16. The preparation of claim 15, wherein the preparation comprises, based on a total weight of (a) to (d), from 0.5% to 50% by weight of (a), from 0.5% to 50% by weight of (b), from 0.5% to 50% by weight of (c), and from 0.01% to 50% by weight of (d).

17. The preparation of claim 14, wherein the preparation comprises, based on a total weight of (a) to (d), from 1% to 25% by weight of (a), from 1% to 25% by weight of (b), from 1% to 25% by weight of (c), and from 0.1% to 10% by weight of (d).

18. The preparation of claim 12, wherein the preparation further comprises from 0.001% to 30% by weight of at least one antioxidant, based on a total weight of the preparation.

19. The preparation of claim 13, wherein the preparation further comprises from 0.05% to 20% by weight of at least one antioxidant, based on a total weight of the preparation.

20. The preparation of claim 14, wherein the preparation further comprises from 0.1% to 10% by weight of at least one antioxidant, based on a total weight of the preparation.

21. The preparation of claim 12, wherein the preparation comprises HYDROLYZED COLLAGEN, HYDROLYZED ELASTIN, HYDROLYZED KERATIN, HYDROLYZED SERICIN, and HYDROLYZED SILK.

22. The preparation of claim 12, wherein the preparation is suitable for topical application to at least one of hair and skin.

23. A cosmetic preparation comprising HYDROLYZED COLLAGEN, HYDROLYZED ELASTIN, HYDROLYZED KERATIN, HYDROLYZED SERICIN, and HYDROLYZED SILK.

24. The preparation of claim 23, wherein the preparation comprises from 0.005% to 0.03% by weight of HYDROLYZED COLLAGEN, from 0.005% to 0.03% by weight of HYDROLYZED ELASTIN, from 0.05% to 0.5% by weight of HYDROLYZED KERATIN, from 0.01% to 0.5% by weight of HYDROLYZED SERICIN, and from 0.01% to 0.5% by weight of HYDROLYZED SILK, in each case based on a total weight of the preparation.

25. The preparation of claim 24, wherein the preparation further comprises from 0.001% to 30% by weight of at least one antioxidant, based on a total weight of the preparation.

26. The preparation of claim 12, wherein the preparation is present as at least one of a hydrous preparation, an emulsion, a gel, a hydrodispersion, an aqueous surfactant solution, a microemulsion.

27. A hair care composition which comprises the preparation of claim 12.

28. A shampoo which comprises the preparation of claim 12.

29. A hair care composition which comprises the preparation of claim 23.

30. A method of cleansing or caring for at least one of hair and scalp, wherein the method comprises applying to at least one of hair and scalp the preparation of claim 12.

31. A method of cleansing or caring for at least one of hair and scalp, wherein the method comprises applying to at least one of hair and scalp the preparation of claim 23.
